(19) United States
(12) Patent Application Publication
(10) Pub. No.: US 2013/0165522 A1
(43) Pub. Date: Jun. 27, 2013

(54) GEL FORMULATIONS FOR THE TOPICAL USE OF 1-AMINO-ALKYLCYCLOHEXANE DERIVATIVES

(71) Applicants: Petra Scheppler, Mainz (DE); Bernhard Hauptmeier, Gelnhausen (DE); Alexander Linko, Frankfurt am Main (DE); Harry Abts, Oberursel (DE)

(72) Inventors: Petra Scheppler, Mainz (DE); Bernhard Hauptmeier, Gelnhausen (DE); Alexander Linko, Frankfurt am Main (DE); Harry Abts, Oberursel (DE)

(73) Assignee: MERZ PHARMA GmbH & CO. KGaA, FRANKFURT am MAIN (DE)

(21) Appl. No.: 13/704,065

(22) PCT Filed: Jun. 17, 2011
(86) PCT No.: PCT/EP11/03023

§ 371 Date: Feb. 15, 2013

Related U.S. Application Data
(60) Provisional application No. 61/356,073, filed on Jun. 18, 2010.
(60) Provisional application No. 61/356,101, filed on Jun. 18, 2010.
(60) Provisional application No. 61/402,190, filed on Aug. 25, 2010.
(60) Provisional application No. 61/402,164, filed on Aug. 25, 2010.

(30) Foreign Application Priority Data
Jun. 18, 2010 (EP) 10006364.3
Jun. 18, 2010 (EP) 10006365.0

(51) Int. Cl. A61K 31/13

(52) U.S. Cl. CPC A61K 31/13
USPC 514/579

(57) ABSTRACT

This invention provides a pharmaceutical composition for topical application to the skin of a patient in need of, comprising neramexane or a pharmaceutically acceptable salt thereof, and at least one gel forming agent for the treatment or prevention of inflammatory skin diseases such as impetigo contagiosa, acne, rosacea, eczema, atopic dermatitis, contact dermatitis, seborrheic dermatitis, psoriasis, and/or oily skin. The at least one gel forming agent comprises polymers with neutral and/or positively charged polymer backbones.

### FIELD OF THE INVENTION

This invention provides a pharmaceutical composition for topical application to the skin of a patient in need of, comprising neramexane or a pharmaceutically acceptable salt thereof, and at least one gel forming agent for the treatment or prevention of inflammatory skin diseases such as impetigo contagiosa, acne, rosacea, eczema, atopic dermatitis, contact dermatitis, seborrheic dermatitis, psoriasis, and/or oily skin. The at least one gel forming agent comprises polymers with neutral and/or positively charged polymer backbones.

### BACKGROUND OF THE INVENTION

Neramexane (1-amino-1,3,3,5,5-pentamethylcyclohexane) and other 1-amino-alkylcyclohexane derivatives are used for therapies of various diseases, especially neurological diseases, including Alzheimer's disease and neuropathic pain. Neramexane and other 1-amino-alkylcyclohexane derivatives are disclosed in detail in U.S. Pat. Nos. 6,034,134 and 6,071,966, the subject matter of these patents being hereby incorporated by reference.

WO 2005/044228 discloses an aqueous based formulation comprising cyclohexylamine or aminoadamantane, and being optionally free of preservatives.

WO2007/062815 discloses special modified release dosage forms comprising neramexane, which may be useful for treating diabetic neuropathic pain, amyotrophic lateral sclerosis, multiple sclerosis, irritable bowel syndrome, appetite disorders, obesity, binge eating disorders, autism, attention deficit syndrome, attention deficit hyperactivity disorder, bipolar disorder, tinnitus, mycosis, psoriasis, and other conditions.

It has now been found that 1-amino-alkylcyclohexane derivatives such as neramexane are also suitable for treating numerous inflammatory skin diseases, since it can be shown that neramexane exhibits antibacterial activity against bacteria related to acne, such as Propionibacterium acnes, against Staphylococcus aureus, which is relevant for atopic dermatitis and the skin disease impetigo contagiosa, and, to an even greater extent, against Streptococcus pyogenes, which is also involved in impetigo contagiosa.

Inflammatory skin diseases include, for example, impetigo contagiosa, acne, rosacea, eczema, atopic dermatitis, contact dermatitis, seborrheic dermatitis, psoriasis, and oily skin.

Inflammatory skin diseases can have a strong influence on the quality of life of patients. These diseases can be physically uncomfortable, they can cause unsightly lesions and severe scarring, thereby decreasing self-esteem and self-confidence, and permanently impact life.

Acne is the most common inflammatory skin disease. Epidemiologic data suggest that up to 80% of individuals may be affected. Men and women develop acne about equally, and the onset of the disease typically occurs 10-14 years of age and regresses at 20-25 years of age. In some patients acne persists into the fourth or fifth decade of life (persistent acne). The clinical spectrum of acne ranges from mild manifestations (e.g., a few acne lesions with occasional inflamed papulopustules) to “clinical” acne with severe inflammation and abscess formation on the face or upper trunk. Follicular rupture may follow leading to a foreign body reaction including abscesses, fistulas, and systemic signs of inflammation (acne conglobata). It is estimated that twenty percent of all Americans afflicted with acne will have severe acne, which results in permanent physical scarring.

Propionibacterium acnes (P.acnes) is the most common gram-positive microaerophilic organism found on normal skin. Although it has no intrinsic pathogenicity, P.acnes is believed to play a major role in the pathogenesis of acne. Most presently available topical anti-acne preparations such as benzoyl peroxides and topical antibiotics exert their therapeutic effect through inhibition or elimination of P.acnes.

There are several different types of acne. Acne vulgaris, an inflammatory disease of the pilosebaceous glands is characterized by an eruption of the skin. It is a common affliction of the adolescent and affects a small percentage of the adult population. Acne rosacea is characterized by erythema with or without an acneiform component. Rosacea typically occurs in adults of about 30-50 years of age.

Four factors are generally believed to be the main contributors to the onset of acne disease: an increased sebum production; faulty desquamation leading to formation of comedo, increased colonization of the pilosebaceous duct by anaerobic Propionibacterium acnes; and an inflammatory response.

Various topical and systemic agents are utilized in the treatment of acne and other inflammatory skin diseases including various scrubbing or abrasive compositions, deep cleaning or astringent compositions, and exposure to ultraviolet radiation. Current treatments comprise benzoyl peroxide, oral and topical bacteriostatics, topical and systemic antibiotics, retinoids, antiseborrheic medications, salicylic acid, alpha hydroxyl acid, azelaic acid, nicotinamide, exfoliants, medolytics, keratolytic soaps, steroids and other immunosuppressive agents, as well as hormones for female patients.

While these therapies for inflammatory skin diseases may provide improvement for some patients, there are various disadvantages associated with the available treatments.

For example, steroid therapy may have an undesirable effect on appearance by causing weight gain, blotting, and puffiness. Additionally, steroids and other immunosuppressants can place patients at increased risk for infection. Retinoids may act as irritants and are also known to possess teratogenic properties. Long term antibiotic treatment can promote the emergence of antibiotic resistance. Thus, there exists a need for improved treatments for inflammatory skin diseases, like acne.

Neramexane and other 1-amino-alkylcyclohexane derivatives are suitable for the treatment of various inflammatory skin diseases, including acne. Increased sebum production, the oily substance of the sebaceous glands, is believed to be regulated by androgen hormones, and thought to be one of the main causes of acne (seborrhea) development. The beneficial impact of neramexane and other 1-amino-alkylcyclohexane derivatives on sebocytes, cells of the sebaceous glands, contributes to its effective treatment of acne and various other inflammatory skin diseases. The impact on proliferation and/or differentiation of sebocytes and thus, the ability to reduce lipid production, allows for regulation of sebum secretion. In addition to overall sebum regulation, the composition of sebum may also be influenced by neramexane, leading to normalization of the pathophysiological phenotype of the diseased hair-follicle.

Patients with inflammatory skin diseases often exhibit also a disturbed cutaneous barrier function. 1-Amino-alkylcyclohexane derivatives, such as neramexane, may improve the cutaneous barrier function and block the delay of barrier recovery, which results in a positive effect on homeostasis of the skin.

As of yet, no satisfactory formulation comprising neramexane for the treatment of inflammatory skin diseases exists, such as a gel formulation for topical application. A common problem of manufacturing topical pharmaceutical gel formulations is the compatibility of suitable gel forming agent(s) with therapeutically effective amounts of the active ingredient(s). Due to chemical characteristics, gel forming agents are either not compatible with neramexane, or only allow the combination of limited, non-therapeutically effective amounts of this ingredient. Besides the requirement of the formulation to incorporate sufficient active ingredient, the release of the active ingredient from the formulation, and the long-term storage of the formulation in good quality, are also important necessities for manufacturing.

The current state of the art for manufacturing topical gel formulations, comprises the use of carbomer gel forming agents. These gel forming agents are limited in their capability to be combined with neramexane since they only allow the use of limited amounts thereof. Moreover, the gel structure of a carbomer gel, such as polyacrylate, can be disturbed or destructed by high electrolyte compounds, especially in high concentration. Carbomer gels can further reduce the capacity of emulgators and since most polyacrylates are synthetic they can cause skin irritations, which makes them challenging for the use in formulations for the treatment of skin diseases.

Based on the problems discussed above in regard to the prior art, one object according to the present invention is to provide a pharmaceutical composition in form of a gel formulation, comprising neramexane or a pharmaceutically acceptable salt thereof, for the treatment or prevention of an inflammatory skin disease.

This object and others are solved by testing and evaluating various gel forming agents for their capability to generate one or more applicable gel formulation(s) comprising neramexane exhibiting with good dermal and systemic bioavailability, good properties to incorporate and to release therapeutically effective amounts of neramexane, sufficient penetration and storage capacities, and finally with the properties to not burden the body with excessive penetration and systemic delivery of the active ingredient.

The technical problems are solved by the embodiments characterized in the claims and herein below.

### OBJECTS OF THE INVENTION

The first object of the present invention was to provide a pharmaceutical composition for topical application to the skin of a subject, comprising a 1-amino-alkylcyclohexane derivative, particularly neramexane or a pharmaceutically acceptable salt thereof.

Another object of the present invention was to provide a pharmaceutical composition wherein said pharmaceutical composition is for the treatment or prevention of neuropathic pain or a skin disease, particularly for the treatment or prevention of an inflammatory skin disease, particularly taken from the list of: impetigo contagiosa, acne, rosacea, eczema, atopic dermatitis, contact dermatitis, seborrheic dermatitis, psoriasis, and oily skin.

Another object of the present invention was to provide a kit comprising a pharmaceutical composition of a 1-amino-alkylcyclohexane derivative, particularly neramexane or a pharmaceutically acceptable salt thereof, and a patch for positioning said pharmaceutical composition on a defined region of the skin of a patient.

### SUMMARY OF THE INVENTION

In a first aspect, the present invention relates to a pharmaceutical composition for topical application to the skin of a patient comprising
a) a 1-amino-alkylcyclohexane derivative, particularly neramexane or a pharmaceutically acceptable salt thereof; and
b) at least one gel forming agent, wherein said gel forming agent is a polymer having a neutral or positively charged polymer backbone.

In a second aspect, the present invention relates to the use of a 1-amino-alkylcyclohexane derivative, particularly neramexane or a pharmaceutically acceptable salt thereof, for the preparation of a pharmaceutical composition as medicament for topical application to the skin of a patient, wherein the pharmaceutical composition additionally comprises at least one gel forming agent, wherein said gel forming agent is a polymer having a neutral or positively charged polymer backbone.

In a third aspect, the present invention relates to a method of treating a patient in need thereof by topically applying to the skin of said patient a pharmaceutical composition comprising
a) a 1-amino-alkylcyclohexane derivative, particularly neramexane or a pharmaceutically acceptable salt thereof; and
b) at least one gel forming agent, wherein said gel forming agent is a polymer having a neutral or positively charged polymer backbone.

In particular embodiments of the invention, said at least one gel forming agent is selected from the list of: chitosan, Xanthan gum, cellulose, and hydroxyalkylcellulose, particularly hydroxyethylcellulose or hydroxypropylcellulose.

In particular embodiments of the invention, said pharmaceutical composition comprises a mixture of at least two different gel forming agents comprising
a) at least one polymer having a neutral polymer backbone; and
b) at least one polymer having a positively charged polymer backbone.

In particular embodiments of the invention, said at least one polymer having a neutral polymer backbone is cellulose or a hydroxyalkylcellulose, particularly a hydroxyethylcellulose or a hydroxypropylcellulose, most particularly a hydroxypropylcellulose, and wherein said at least one polymer having a positively charged polymer backbone is chitosan.

In particular embodiments of the invention, said at least one polymer having a neutral polymer backbone and said at least one polymer having a positively charged polymer backbone are present in said mixture in a ratio of between 1 to about 0.04 and 1 to about 25, particularly in a ration between 1 to about 0.1 and 1 to about 9.

In particular embodiments of the invention, said at least one gel forming agent is present in a topical formulation at a concentration of between about 0.5% and about 5%.

In particular embodiments of the invention, said pharmaceutical composition is for the treatment or prevention of neuropathic pain or a skin disease, particularly for the treatment or prevention of an inflammatory skin disease, particularly taken from the list of: impetigo contagiosa, acne, rosacea, eczema, atopic dermatitis, contact dermatitis, seborrheic dermatitis, psoriasis, and oily skin.

In particular embodiments of the invention, said neramexane is present as neramexane mesylate.

In particular embodiments of the invention, said concentration of neramexane, or of neramexane contained in the pharmaceutically acceptable salt thereof, is within a range of about 0.1% to about 20%, particularly of about 0.5% to about 10%, particularly of about 1% to about 8%, or of about 8% to about 20%.

In particular embodiments of the invention, said concentration of neramexane, or of neramexane contained in the pharmaceutically acceptable salt thereof, is within a range of about 0.5% to about 10%, or within a range of about 0.5% to about 3%, particularly at about 0.5%, at about 1.5% or at about 3%, in each case by weight of the total composition.

In particular embodiments of the invention, said pharmaceutical composition further comprises at least one solvent, particularly a hydrophilic solvent.

In particular embodiments of the invention, said solvent is present in a concentration of about 70% to about 96.5% by weight of the total composition.

In particular embodiments of the invention, said solvent is present in a concentration of between about 93% and about 96.5% by weight of the total composition.

In particular embodiments, the solvent is water.

In particular embodiments of the invention, the pH is adjusted to a pH value between about 4.5 and about 6.5, particularly to about 5.5, particularly wherein said pharmaceutical composition further comprises at least one buffer system.

In particular embodiments of the invention, a kit comprises the pharmaceutical composition, as well as a patch for positioning said pharmaceutical composition on a defined region of the skin of a patient.

### BRIEF DESCRIPTION OF THE DRAWING

The invention disclosed herein is best understood from the detailed description when read in conjunction with the following accompanying figure.

FIG. 1 shows an overview of a dermal bioavailability test of 13 prototype formulations, tested in vitro on fresh human skin. Using the tape-strip technique and C14-labeled neramexane, individual cell layers of the stratum corneum, the epidermis, and the dermis were analyzed for their content of neramexane. Quantification of neramexane was performed using liquid-scintillation counting.

FIG. 2 shows the ACNE map for a clinical study, including the four facial areas for administration of the investigational product.

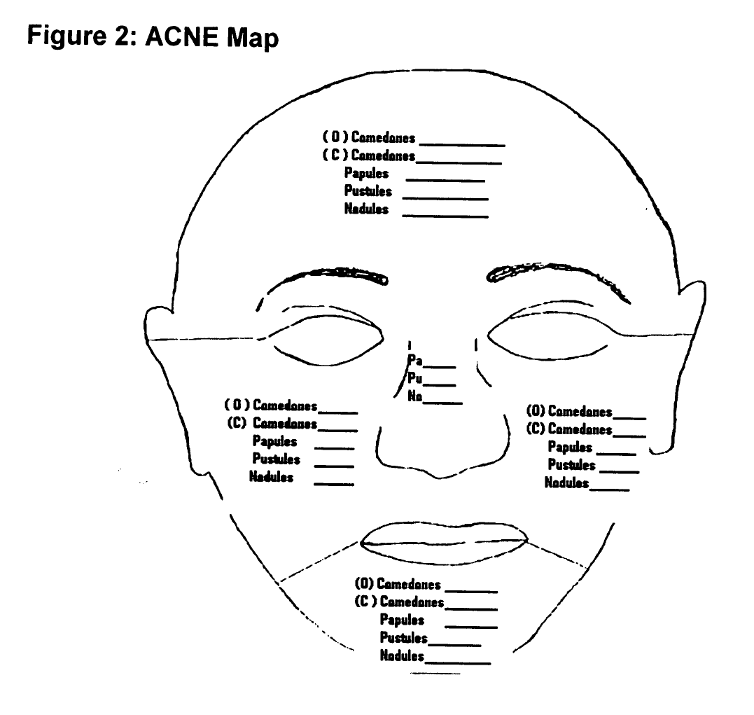

### DETAILED DESCRIPTION OF THE INVENTION

In a first aspect, the present invention relates to a pharmaceutical composition for topical application to the skin of a patient comprising
a. a 1-amino-alkylcyclohexane derivative, particularly neramexane or a pharmaceutically acceptable salt thereof; and
b. at least one gel forming agent, wherein said gel forming agent is a polymer having a neutral or positively charged polymer backbone.

In a second aspect, the present invention relates to the use of a 1-amino-alkylcyclohexane derivative, particularly neramexane or a pharmaceutically acceptable salt thereof, for the preparation of a pharmaceutical composition as medicament for topical application to the skin of a patient, wherein the pharmaceutical composition additionally comprises at least one gel forming agent, wherein said gel forming agent is a polymer having a neutral or positively charged polymer backbone.

In a third aspect, the present invention relates to a method of treating a patient in need thereof by topically applying to the skin of said patient a pharmaceutical composition comprising
a) a 1-amino-alkylcyclohexane derivative, particularly neramexane or a pharmaceutically acceptable salt thereof; and
b) at least one gel forming agent, wherein said gel forming agent is a polymer having a neutral or positively charged polymer backbone.

In the context of the present invention, the term “topical application” refers to applying or spreading the pharmaceutical composition of the present invention onto the surface of an area on the skin of the patient.

As used herein, the term “patient” encompasses mammals including humans and animals, particularly humans.

In the context of the present invention, the term “comprises” or “comprising” means “including, but not limited to”. The term is intended to be open-ended, that specify the presence of any stated features, elements, integers, steps, or components, but do not preclude the presence or addition of one or more other features, elements, integers, steps, components, or groups thereof. The term “comprising” thus includes the more restrictive terms “consisting of” and “consisting essentially of”.

The term 1-amino-alkylcyclohexane derivative is used herein to describe a 1-amino-alkylcyclohexane or a compound derived from 1-amino-alkylcyclohexane, e.g., pharmaceutically acceptable salts of 1-amino-alkylcyclohexanes.

The 1-amino-alkylcyclohexane derivatives of the present invention may be represented by the general formula (I):

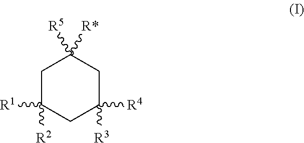

wherein R* is —(CH₂)ₙ—(CR⁶R⁷)ₘ—NR⁸R⁹
wherein n+m=0, 1, or 2
wherein R¹ through R⁷ are independently selected from the group consisting of hydrogen and C₁₋₆alkyl, wherein R⁸ and R⁹ are independently selected from the group consisting of hydrogen and C₁₋₆alkyl or together represent lower-alkylene —(CH₂)ₓ— wherein x is 2 to 5, inclusive, and optical isomers, enantiomers, hydrates, and pharmaceutically-acceptable salts thereof.

Non-limiting examples of the 1-amino-alkylcyclohexanes used according to the present invention include:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane (neramexane),
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane,
N-ethyl-1-amino-1,3,3,5,5-pentamethyl-cyclohexane,
N-(1,3,3,5,5-pentamethylcyclohexyl)pyrrolidine,
3,3,5,5-tetramethylcyclohexylmethylamine,
1-amino-1-propyl-3,3,5,5-tetramethylcyclohexane,
1 amino-1,3,3,5(trans)-tetramethylcyclohexane (axial amino group),
3-propyl-1,3,5,5-tetramethylcyclohexylamine semihydrate,
1-amino-1,3,5,5-tetramethyl-3-ethylcyclohexane,
1-amino-1,3,5-trimethylcyclohexane,
1-amino-1,3-dimethyl-3-propylcyclohexane,
1-amino-1,3(trans),5(trans)-trimethyl-3(cis)-propylcyclohexane,
1-amino-1,3-dimethyl-3-ethylcyclohexane,
1-amino-1,3,3-trimethylcyclohexane,
cis-3-ethyl-1(trans)-3(trans)-5-trimethylcyclohexamine,
1-amino-1,3(trans)-dimethylcyclohexane,
1,3,3-trimethyl-5,5-dipropylcyclohexylamine,
1-amino-1-methyl-3(trans)-propylcyclohexane,
1-methyl-3(cis)-propylcyclohexylamine,
1-amino-1-methyl-3(trans)-ethylcyclohexane,
1-amino-1,3,3-trimethyl-5(cis)-ethylcyclohexane,
1-amino-1,3,3-trimethyl-5(trans)-ethylcyclohexane,
cis-3-propyl-1,5,5-trimethylcyclohexylamine,
trans-3-propyl-1,5,5-trimethylcyclohexylamine,
N-ethyl-1,3,3,5,5-pentamethylcyclohexylamine,
N-methyl-1-amino-1,3,3,5.5-pentamethylcyclohexane,
1-amino-1-methylcyclohexane,
N,N-dimethyl-1-amino-1,3,3,5,5-pentamethylcyclohexane,
2-(3,3,5,5-tetramethylcyclohexyl)ethylamine,
2-methyl-1-(3,3,5,5-tetramethylcyclohexyl)propyl-2-amine,
2-(1,3,3,5,5-pentamethylcyclohexyl-1)-ethylamine semihydrate,
N-(1,3,3,5,5-pentamethylcyclohexyl)-pyrrolidine,
1-amino-1,3(trans),5(trans)-trimethylcyclohexane,
1-amino-1,3(cis),5(cis)-trimethylcyclohexane,
1-amino-(1R,5S)trans-5-ethyl-1,3,3-trimethylcyclohexane,
1-amino-(1S,5S)cis-5-ethyl-1,3,3-trimethylcyclohexane,
1-amino-1,5,5-trimethyl-3(cis)-isopropyl-cyclohexane,
1-amino-1,5,5-trimethyl-3(trans)-isopropyl-cyclohexane,
1-amino-1-methyl-3(cis)-ethyl-cyclohexane,
1-amino-1-methyl-3(cis)-methyl-cyclohexane,
1-amino-5,5-diethyl-1,3,3-trimethyl-cyclohexane,
1-amino-1,3,3,5,5-pentamethylcyclohexane,
1-amino-1,5,5-trimethyl-3,3-diethylcyclohexane,
1-amino-1-ethyl-3,3,5,5-tetramethylcyclohexane,
N-ethyl-1-amino-1,3,3,5,5-pentamethylcyclohexane,
N-(1,3,5-trimethylcyclohexyl)pyrrolidine or piperidine,
N-[1,3(trans),5(trans)-trimethylcyclohexyl]pyrrolidine or piperidine,
N-[1,3(cis),5(cis)-trimethylcyclohexyl]pyrrolidine or piperidine,
N-(1,3,3,5-tetramethylcyclohexyl)pyrrolidine or piperidine,
N-(1,3,3,5,5-pentamethylcyclohexyl)pyrrolidine or piperidine,
N-(1,3,5,5-tetramethyl-3-ethylcyclohexyl)pyrrolidine or piperidine,
N-(1,5,5-trimethyl-3,3-diethylcyclohexyl)pyrrolidine or piperidine,
N-(1,3,3-trimethyl-cis-5-ethylcyclohexyl)pyrrolidine or piperidine,
N-[(1S,5S)cis-5-ethyl-1,3,3-trimethylcyclohexyl]pyrrolidine or piperidine,
N-(1,3,3-trimethyl-trans-5-ethylcyclohexyl)pyrrolidine or piperidine,
N-[(1R,5S)trans-5-ethyl,3,3-trimethylcyclohexyl]pyrrolidine or piperidine,
N-(1-ethyl-3,3,5,5-tetramethylyclohexyl)pyrrolidine or piperidine,
N-(1-propyl-3,3,5,5-tetramethylcyclohexyl)pyrrolidine or piperidine,
N-(1,3,3,5,5-pentamethylcyclohexyl)pyrrolidine,
and optical isomers, diastereomers, enantiomers, hydrates, their pharmaceutically acceptable salts, and mixtures thereof.

1-Amino-alkylcyclohexane derivatives (e.g., neramexane, 1-amino-1,3,3,5,5-pentamethylcyclohexane) are disclosed in U.S. Pat. Nos. 6,034,134 and 6,071,966. 1-Amino-alkylcyclohexane derivatives (e.g., neramexane) may be used according to the invention in the form of any of pharmaceutically acceptable salts, solvates, isomers, conjugates, and prodrugs, any references to 1-amino-alkylcyclohexane derivatives (e.g., neramexane) in this description should be understood as also referring to such salts, solvates, isomers, conjugates, and prodrugs.

The term “analog” or “derivative” is used herein in the conventional pharmaceutical sense, to refer to a molecule that structurally resembles a reference molecule (such as neramexane), but has been modified in a targeted and controlled manner to replace one or more specific substituents of the reference molecule with an alternate substituent, thereby generating a molecule which is structurally similar to the reference molecule. Synthesis and screening of analogs (e.g., using structural and/or biochemical analysis), to identify slightly modified versions of a known compound which may have improved or biased traits (such as higher potency and/or selectivity at a specific targeted receptor type, greater ability to penetrate mammalian barriers, such as cell membranes, fewer side effects, etc.) is a drug design approach that is well known in pharmaceutical chemistry.

The term “treat” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

In the context of the present invention, the term “pharmaceutically acceptable salts” includes, but is not limited to, acid addition salts, such as those made with hydrochloric, methylsulfonic, hydrobromic, hydroiodic, perchloric, sulfuric, nitric, phosphoric, acetic, propionic, glycolic, lactic, pyruvic, malonic, succinic, fumaric, tartaric, citric, benzoic, carbonic, cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid. All of these salts (or other similar salts) may be prepared by conventional means. The nature of the salt is not critical, provided that it is non-toxic and does not substantially interfere with the desired pharmacological activity.

The term “gel” as used herein, refers in a broad sense to a semi-solid system having a solid phase dispersed in a liquid phase, wherein the solid phase is the continuous phase and the liquid is the discontinuous phase. The particles forming the solid phase are no longer independent kinetic units, but are spatially fixed due to a particular structural arrangement, such as by forming secondary connections, e.g. van der Waal's interactions or hydrogen bonds. It is intended to represent the physical, gelatinous characteristic of the composition rather than being limited to a restrictive, technical definition.

The term “gel forming agent” as used herein refers to various gelling and viscosity agents, solution binders, thickeners, emulsifiers. In a preferred embodiment, the thickening agent is employed in an amount effective to form a semi-solid that is substantially translucent and is sufficiently viscous. Gel forming agents include agents, which form a semi-crystalline structure by reaction with another material or by lowering of the temperature thereof while dissolved or colloidally suspended in a liquid medium. Gels can be either formed with a single or with a mixture of gel forming agents. The term “mixture” as used herein, refers to multiple gel forming agents. Gel formation takes place within from about 2 minutes to about 16 hours after mixing, depending upon the components utilized. The gel-form pharmaceutical compositions of the present invention may be formulated by conventional mixing of the components described in the examples.

The term “neutral polymer backbone” as used herein refers to polymers, where the polymer backbone carries either no charged residues, or a balanced number of both positively and negatively charged residues.

The term “positively charged polymer backbone” as used herein refers to cationic polymers, i.e. polymers where the polymer backbone carries positively charged residues. The positive charges prevent the formation of coiled polymers, allowing to contribute for better viscosity in their stretched state, because the stretched-out polymer takes up more space than a coiled polymer and this resists the flow of solvent molecules around it.

In particular embodiments of the invention, said at least one gel forming agent is selected from the list of: chitosan, Xanthan gum, cellulose, and hydroxyalkylcellulose, particularly hydroxyethylcellulose or hydroxypropylcellulose. In particular embodiments, said at least one gel forming agent is either chitosan or hydroxypropylcellulose.

Chitosan is a linear polysaccharide composed of β-(1-4)-linked D-glucoseamine and N-acetyl-D-glucosamine moieties. Due to the free amino groups, chitosan is either neutral or a cationic polymer, depending on the pH value of the environment. Chitosan is a deacetylated derivative of chitin, usually isolated from the shells of crustaceans, and used as a gelling agent. An example of a chitosan in accordance with the present invention is Chitopharm®, such as Chitopharm® M (molecular weight range: 100 to 2,000 kDa; degree of deacetylation: at least 70%; US Drug Master File No. 19245), or Chitopharm® L (molecular weight range: 500 to 5,000 kDa; degree of deacetylation: at least 70%; US Drug Master File No. 19244).

An example of a Xanthan gum in accordance with the present invention is Xantural 75. It is known for its strong ability to increase the viscosity of liquids and therefore, widely used as thickening agent.

An example of a hydroxyethylcellulose in accordance with the present invention is Natrosol, such as Natrosol Pharm 250. An example of a hydroxypropylcellulose in accordance with the present invention is Klucel, such as Klucel MF Pharma. It is known and applied as a solution binder.

In particular embodiments of the invention, said pharmaceutical composition comprises a mixture of at least two different gel forming agents comprising
a) at least one polymer having a neutral polymer backbone; and
b) at least one polymer having a positively charged polymer backbone.

In particular embodiments of the invention, said at least one polymer having a neutral polymer backbone is cellulose or a hydroxyalkylcellulose, and wherein said at least one polymer having a positively charged polymer backbone is chitosan.

In particular embodiments of the invention, said at least one polymer having a neutral polymer backbone and said at least one polymer having a positively charged polymer backbone are present in said mixture in a ratio of between 1 to about 0.04 and 1 to about 25, particularly in a ration between 1 to about 0.1 and 1 to about 9.

In the context of the present invention, the term “about” or “approximately” means within 20%, alternatively within 10%, including within 5% of a given value or range. Alternatively, especially in biological systems, the term “about” means within about a log (i.e., an order of magnitude), including within a factor of two of a given value.

All percentages and ratios used herein are by weight, unless otherwise specified.

In particular embodiments of the invention, said at least one gel forming agent is present in a concentration of between about 0.5% and about 5%.

In particular embodiments of the invention, said pharmaceutical composition is for the treatment or prevention of neuropathic pain or a skin disease.

In particular embodiments, the skin disease is an inflammatory skin disease.

In the context of the present invention, the term “inflammatory skin diseases” refers to diseases characterized by a series of clinical signs and symptoms, such as itch, edema, erythema and abrasion, which are induced by various stimulative factors that cause a series of inflammatory reactions in the skin epithelium. The inflammatory skin diseases are characterized by the occurrence of a skin lesion resulting from activation of Langerhans-cells, infiltration of inflammatory cells such as activated helper T cells and monocytes, and accompanying acanthosis, or abnormal differentiation of keratinocytes. According to the present invention, inflammatory skin diseases comprise impetigo contagiosa, acne, eczema, atopic dermatitis, rosacea, contact dermatitis, seborrheic dermatitis, psoriasis, oily skin, lichen planus, pityriasis rubra pilaris, and palmoplanter pustulosis, but are not limited thereto.

In particular embodiments, the inflammatory skin disease is taken from the list of: impetigo contagiosa, acne, rosacea, eczema, atopic dermatitis, contact dermatitis, seborrheic dermatitis, psoriasis, and oily skin.

As used herein, the term impetigo contagiosa includes bullous impetigo and ecthyma.

As used herein, the term “acne” includes all types of acne in all stages, including acne vulgaris observed in adolescents, persistent acne, clinical acne, acne observed in endocrinologic conditions characterized by excess androgen secretion, and the like, in the active inflammatory (pustule-, papule-, comedone-forming) and non-inflammatory (blackhead- and cyst-forming) phases, and post-inflammatory (healing, scarring, and scarred) phase.

In a particular embodiment, the present invention relates to the treatment of subjects with moderate to severe acne with inflammatory and non-inflammatory lesions, and to pharmaceutical compositions for use in the treatment of subjects with moderate to severe acne with inflammatory and non-inflammatory lesions.

In certain such embodiments, a subject to be treated is selected based on the clinical presentation of the subject being diagnosed with facial acne and a combination of inflammatory and non-inflammatory lesions.

In certain such embodiments, neramexane mesylate is in a topical gel formulation for an application twice daily for 12 weeks.

As used herein, the term rosacea includes persistent edema of rosacea, rosacea conglobata, rosacea fulminans, ophthalmic rosacea, lupoid or granulomatous rosacea, steroid rosacea, gram-negative rosacea, halogen rosacea, phymas in rosacea, erythematotelangiectatic rosacea, papulopustular rosacea, phymatous rosacea and ocular rosacea.

As used herein, the term eczema includes atopic eczema, irritant contact dermatitis, allergic contact dermatitis, occupational dermatitis, xerotic eczema, seborrhoeic dermatitis, syshidrosis, discoid eczema, venous eczema, dermatitis herpetiformis, neurodermatitis, and autoeczematization.

As used herein, the term psoriasis includes psoriasis vulgaris, plaque psoriasis, flexural psoriasis, inverse psoriasis, guttate psoriasis, pustular psoriasis, nail psoriasis, erythrodermic psoriasis and psoriatic arthritis.

In particular embodiments of the invention, said neramexane is present as neramexane mesylate.

In particular embodiments of the invention, said neramexane is present as neramexane mesylate hydrate.

In particular embodiments of the invention, the concentration of neramexane mesylate is within a range of about 0.1% to about 20%, particularly of about 0.5% to about 10%, particularly of about lcY0 to about 8%, or of about 8% to about 20% (w/w).

In particular embodiments of the invention, said concentration of neramexane, or of neramexane contained in the pharmaceutically acceptable salt thereof, is within a range of about 0.5% to about 10%, or within a range of about 0,5% to about 3%, particularly at about 0.5%, at about 1.5% or at about 3%, in each case by weight of the total composition.

In embodiments, where free neramexane, or a pharmaceutically acceptable salt other than the mesylate, is used, the concentration will be adjusted accordingly. For example, a concentration of 10% (w/w) of neramexane mesylate (molecular weight: 265.42 g/mol) corresponds to a concentration of 6.4% (w/w) of free neramexane (molecular weight: 169.31 g/mol).

In particular embodiments of the invention, said pharmaceutical composition further comprises at least one solvent, particularly a hydrophilic solvent.

In the context of the present invention, the term “solvent” refers to a liquid that is suitable as liquid phase for dispersion of the solid phase for gel formation. The solvent may be a single liquid or a mixture or two or more liquids. In particular embodiments, the liquid is water.

In particular embodiments of the invention, said solvent is present in a concentration of about 70% to about 96.5% by weight of the total composition.

In particular embodiments of the invention, said solvent is present in a concentration of between about 93% and about 96.5% by weight of the total composition.

In particular embodiments of the invention, the pH is adjusted to a pH value between about 4.5 and about 6.5, particularly to about 5.5, particularly wherein said pharmaceutical composition further comprises at least one buffer system.

The term “buffer” or “buffer system” as used herein, refers to a compound or combination of compounds that adjust(s) and maintain the pH of the environment in which is/they is/are dissolved or dispersed to a pH range defined by the buffer or buffer system used. In particular embodiments, the buffer is a PBS buffer.

In certain embodiments of the present invention, the pharmaceutical composition further comprises a penetration enhancer. The term “penetration enhancer” as used herein refers to a material that achieves to increase the permeability of a biological membrane (i.e. the skin) towards a pharmaceutical composition, so as to increase the rate and degree at which the drug penetrates through the membrane. The enhanced penetration as effected though the use of such enhancers can be observed, for example, by measuring the rate of diffusion of the drug through animal or human skin using a diffusion cell apparatus. Such a diffusion cell is described by Merritt et al., Diffusion Apparatus for Skin Penetration, 1 J. of Controlled Release 61 (1984), incorporated herein by reference. Penetration enhancers include Arlasolve DMI, propylene glycol, Transcutol P, and Miglyol 812.

In certain embodiments of the present invention, the pharmaceutical composition further comprises an additional pharmaceutical agent (e.g., antimicrobial agents, antibiotics, retinoids or steroids) which has been shown to be effective in treating or preventing inflammatory skin diseases.

In certain embodiments of the present invention, the pharmaceutical composition further comprises a preservative, particularly a preservative selected from the list of potassium sorbate, polyhexanide, parabene (methyl, propyl, butyl, isobutyl), phenoxyethanol, propylene glycol, benzoic acid and benzyl alcohol, particularly potassium sorbate and polyhexanide. In particular embodiments, the concentration of the preservative is between about 0.005% and about 0.5%, particularly at about 0.005%, at about 0.05%, at about 0.1%, at about 0.2%, or at about 0.5%.

In certain such embodiments, the preservative is potassium sorbate. In particular embodiments, potassium sorbate is the sole preservative present in the composition.

In particular embodiments, the concentration of potassium sorbate is between about 0.05% and about 0.25%, particularly between about 0.05% and about 0.2%, particularly at about 0.1% or at about 0.2%.

In certain other embodiments, the preservative is polyhexanide. In particular embodiments, polyhexanide is the sole preservative present in the composition

In particular embodiments, the concentration of polyhexanide is between about 0.005% and about 0.1%, particularly at about 0.005%, at about 0.05% or at about 0.1%.

In other embodiments of the present invention, the pharmaceutical composition comprises no preservative.

In certain embodiments of the present invention, the pharmaceutical composition is for cojoint administration with an additional pharmaceutical agent (e.g., antimicrobial agents, antibiotics, retinoids or steroids).

In certain such embodiments the 1-amino-alkylcyclohexane derivative (e.g., neramexane or a pharmaceutically acceptable salt thereof such as neramexane mesylate) and the additional pharmaceutical agent (e.g., antimicrobial agents, antibiotics, retinoids or steroids) are administered in a single formulation.

In certain embodiments of the present invention, the pharmaceutical composition comprises a therapeutically effective amount of neramexane.

The term “therapeutically effective” applied to dose or amount refers to that quantity of a compound or pharmaceutical composition that is sufficient to result in a desired activity upon administration to a mammal in need thereof, preferably a positive skin appearance and/or feel, to treat the desired disorder, at a reasonable benefit/risk ratio applicable to any medical treatment. In accordance with the subject invention, the therapeutically effective amount is an amount of an active agent, either alone or in combination with other agents, that regulates and/or improves the skin, but where the amount is low enough to avoid serious side effects, i.e., to provide a reasonable benefit to risk ratio, within the scope of sound judgment of the skilled artisan. The total daily usage of a pharmaceutical composition of the invention will be decided by a patient's attending physician within the scope of sound medical judgment.

In certain embodiments, the pharmaceutical composition is selected from a composition consisting of neramexane, particularly neramexane mesylate, particularly neramexane mesylate hydrate, aqua purificata, potassium dihydrogen phosphate, disodium hydrogen phosphate, potassium sorbate and Klucel MF.

In certain embodiments, the pharmaceutical composition is selected from one of the following compositions:
a) neramexane 0.5%: aqua purificata: 95.934%; potassium dihydrogen hosphate: 0.353%; disodium hydrogen phosphate: 0.013%; potassium sorbate: 0.200%; neramexane mesylate: 0.500%; Klucel MF: 3.000%; and
b) neramexane 1,5%: aqua purificata: 94.934%; potassium dihydrogen phosphate: 0.353%; disodium hydrogen phosphate: 0.013%; potassium sorbate: 0.200%; neramexane mesylate: 1.500%; Klucel MF: 3.000%; and
c) neramexane 3%: aqua purificata: 93.434%; potassium dihydrogen phosphate: 0.353%; disodium hydrogen phosphate: 0.013%; potassium sorbate: 0.200%; neramexane mesylate: 3.000%; Klucel MF: 3.000%.

In particular embodiments of the invention, a kit comprises the pharmaceutical composition, as well as a patch for positioning said pharmaceutical composition on a defined region of the skin of a patient.

The term “patch” as used herein refers to a device, which is used to cover and protect the area on the skin of a patient, where the pharmaceutical composition of the present invention is applied to.

The term “treat” or “treatment” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

The pharmaceutical composition of the present invention may be administered as a monotherapy, or in combination with another agent prescribed for the treatment or prevention of inflammatory skin diseases.

Treatment duration may be short-term, e.g., several weeks (for example 8-14 weeks), or long-term until the attending physician deems further administration.

The rate of application and duration of treatment will depend upon the severity and nature of the condition, the response of the particular patient, and related factors within the sound medical judgment of an attending physician or the patient. In general, for the compositions within the compositional ranges noted above, application rates of from about 0.01 mg/cm² to about 25 mg/cm² per day are used. Application can be made once, or preferably several times, daily for periods of a week or more, to relieve the condition being treated.

The present invention is now further exemplified by way of the non-limiting examples recited herein under. One skilled in the art will appreciate that although specific reagents, methods, devices, and conditions are outlined in the following examples, modifications, alternatives, equivalents, and variations can be made to the embodiments which are meant to be encompassed by the spirit and scope of the invention, as well as by the claims. The figures are accordingly, to be regarded in an illustrative, preferred rather than in an exhaustive or restrictive sense.

### EXAMPLES

### Example 1

### Evaluation of Various Prototype Formulations for Compatibility and Storage Properties

Various prototype formulations are generated by dissolving various amounts of neramexane mesylate in water and by adding various gel forming agents in various amounts. Each mixture is homogenized and the pH is adjusted to a pH value of about 5.5 with citric acid, HCl or NaOH. The properties and characteristics of each prototype formulation are evaluated to assess the compatibility of the components.

### Comparative Example 1.1

### Aristoflex AVC

Aristoflex AVC (INCI: Ammonium Acryloyldimethyltaurate/VP Copolymer), a copolymer of ammonium acryloyldimethyltaurate and vinylpyrrolidone, is a viscosity controller and is tested as a gel forming agent in various ratios with neramexane mesylate (see Table 1).

TABLE 1

| | A1 | A2 | A3 | A4 |
|---|---|---|---|---|
| | [% (w/w)] | [% (w/w)] | [% (w/w)] | [% (w/w)] |
| Aqua purificata | 89 | 94 | 96.5 | 98.5 |
| Aristoflex AVC | 1 | 1 | 1 | 1 |
| Neramexane | 10 | 5 | 2.5 | 0.5 |
| mesylate | | | | |
| Sum | 100 | 100 | 100 | 100 |

No homogeneous gel is formed thereof. Hence, neramexane is not compatible with this gel forming agent. Furthermore, Aristoflex AVC or Carbopol are sensitive to electrolytes and also therefore not compatible with neramexane mesylate. No additional experimental storage tests are performed.

### Example 1.2

### Xantural 75

Xantural 75 (INCI: Xanthan gum), known for its strong ability to increase the viscosity of liquids, is also tested as a gel forming agent in various ratios with neramexane mesylate (see Table 2).

TABLE 2

| | X1 | X2 | X3 | X4 |
|---|---|---|---|---|
| | [% (w/w)] | [% (w/w)] | [% (w/w)] | [% (w/w)] |
| Aqua purificata | 88 | 93 | 95.5 | 97.5 |
| Xantural 75 | 2 | 2 | 2 | 2 |
| Neramexane mesylate | 10 | 5 | 2.5 | 0.5 |
| Sum | 100 | 100 | 100 | 100 |

Each homogenized mixture is adjusted to a pH value of about 5.5 using a 10% citric acid solution and gel formation is monitored. Additionally, experimental storage tests for 7 and 25 days are performed using glass and plastic tubes and different incubation temperatures (6° C., room temperature, and 40° C.). The formulations are tested e.g., for consistency, odor, and color. The test results indicate that the gel forming agent Xantural 75 is compatible with neramexane mesylate. (see Table 3).

TABLE 3

| | | +6° C. | Room temperature | +40° C. |
|---|---|---|---|---|
| 7 days | X1 | OK | OK | OK |
| ″ | X2 | OK (opaque)* | OK (opaque)* | separation* |
| ″ | X3 | OK | OK | OK |
| ″ | X4 | OK | OK | OK |
| 25 days | X1 | OK | OK | OK |
| ″ | X2 | OK (opaque)* | OK (opaque)* | separation* |
| ″ | X3 | OK | OK | OK |
| ″ | X4 | OK | OK | OK |

*experiment was performed only once and probably represents an artifact

### Example 1.3

### Klucel MF Pharma

Klucel MF Pharma (INCI: Hydroxypropylcellulose), a solution binder, is tested as a gel forming agent in various ratios with neramexane mesylate (see Table 4).

TABLE 4

| | K1 | K2 | K3 | K4 |
|---|---|---|---|---|
| | [% (w/w)] | [% (w/w)] | [% (w/w)] | [% (w/w)] |
| Aqua purificata | 88 | 93 | 95.5 | 97.5 |
| Klucel MF | 2 | 2 | 2 | 2 |
| Neramexane | 10 | 5 | 2.5 | 0.5 |
| mesylate | | | | |
| Sum | 100 | 100 | 100 | 100 |

Each homogenized mixture is adjusted to pH 5.,5 using a 10% citric acid solution. Additionally, experimental storage tests for 7 and 25 days are performed using glass and plastic tubes, and different incubation temperatures (room temperature, and 40° C.). The resulting gels are of translucent appearance and the test results indicate that the gel forming agent Klucel MF Pharma shows good compatibility with neramexane mesylate (see Table 5). Up to 30% of neramexane mesylate can be incorporated into Klucel MF Pharma-based formulations, which thus are applicable for topical pharmaceutical administration, i.e. to facilitate a study for determination of the maximal-tolerated-dose (MTD) in mini-pigs.

TABLE 5

| | | Room | |
|---|---|---|---|
| | | temperature | +40° C. |
| 7 days | K1 | OK | OK |
| ″ | K2 | OK | OK |
| ″ | K3 | OK | OK |
| ″ | K4 | OK | OK |
| 25 days | K1 | OK | OK |
| ″ | K2 | OK | OK |
| ″ | K3 | OK | OK |
| ″ | K4 | OK | OK |

### Example 1.4

### Chitopharm L

Chitopharm L (INCI: Chitosan), a cationic polymer, is also tested as a gel forming agent in various ratios with neramexane mesylate.

Each homogenized mixture is adjusted to a pH value of about 5.5 and gel formation is monitored. The resulting gels are of clear appearance and the test results indicate that the gel forming agent Chitopharm L shows good compatibility with neramexane mesylate. Up to a ratio of 30% neramexane mesylate is compatible with Chitopharm L and thus, the formulations are applicable for topical pharmaceutical administration.

Furthermore, 3% neramexane mesylate is combined with Chitopharm L, with or without the addition of a penetration enhancer (e.g. Arlasolve DMI, propylene glycol, or Transcutol P) (see Table 6).

TABLE 6

| | C1 | C2 | C3 | C4 |
|---|---|---|---|---|
| | [% (w/w)] | [% (w/w)] | [% (w/w)] | [% (w/w)] |
| Aqua purificata | 94.44 | 89.44 | 89.44 | 89.44 |
| Chitopharm L | 1.7 | 1.7 | 1.7 | 1.7 |
| Lactic acid | 0.86 | 0.86 | 0.86 | 0.86 |
| Neramexane | 3 | 3 | 3 | 3 |
| mesylate | | | | |
| Propylene glycol | | 5 | | |
| Arlasolve DMI | | | 5 | |
| Transcutol P | | | | 5 |
| Sum | 100 | 100 | 100 | 100 |

Experimental storage tests for several months are performed using glass and plastic tubes, and different incubation temperatures (room temperature and 40° C.). The formulations are tested for viscosity, consistency, odor, and color (see Table 7).

TABLE 7

| | C1 | C2 | C3 | C4 |
|---|---|---|---|---|
| pH start | 5.57 | 5.45 | 5.43 | 5.55 |
| pH 2 months RT | 5.16 | 5.18 | 5.15 | 5.17 |
| pH 7 months RT | 4.75 | 4.79 | 4.78 | 4.80 |
| pH 2 months 40° C. | 4.58 | 4.61 | 4.61 | 4.62 |
| pH 4 months 40° C. | 4.51 | 4.53 | 4.54 | 4.55 |
| Viscosity start [mPa] | 6309 | 6145 | 5993 | 6947 |
| Viscosity 2 months | 4475 | 5208 | 4329 | 4943 |
| RT [mPa] | | | | |
| Viscosity 7 months | 3516 | 4443 | 3052 | 3896 |
| RT [mPa] | | | | |
| Viscosity 2 months | 3455 | 4595 | 2335 | 3640 |
| 40° C. [mPa] | | | | |
| Viscosity 4 months | 2792 | 4210 | 1656 | 2672 |
| 40° C. [mPa] | | | | |
| Appearance in tube at | smell | smell | smell | smell |
| RT after 2 months | acceptable | acceptable | acceptable | acceptable |
| Appearance in tube at | smell | smell | faint smell | smell |
| 40° C. after 2 months | acceptable | acceptable | | acceptable |
| Appearance in glass at | fishy smell | fishy smell | fishy smell; | fishy |
| RT after 2 months | | | faint yellow | smell |
| | | | colour | |
| Appearance in glass at | fishy smell | fishy smell | fishy smell; | fishy smell |
| 40° C. after 2 months | | | faint yellow | |
| | | | colour | |

### Comparative Example 1.5

### Sepineo P 600

Sepineo P 600, a concentrated dispersion of Acrylamide/Sodium Acryloyldimethyl taurate copolymer/Isohexadecane/Polysorbat 80 has self-gelling and thickening properties and the ability to emulsify oily phases.

It is tested in various ratios with neramexane mesylate. Each homogenized mixture is adjusted to a pH value of about 5.5 and gel formation is monitored, resulting in white creamgels. Yet, the formulations are limited in containing higher concentrations of neramexane mesylate since Sepineo P 600 comprises acryloyldimethyl taurate building blocks resulting in a negatively charged backbone. The maximum neramexane concentration that can be obtained is 3%. Concentrations above 3% result in the separation of the white creamgels.

Furthermore, 3% neramexane mesylate is combined with Sepineo P 600, with or without the addition of a penetration enhancer (e.g. Arlasolve DMI, propylene glycol, or Miglyol 812). (see Table 8).

TABLE 8

| | S1 | S2 | S3 | S4 |
|---|---|---|---|---|
| | [% (w/w)] | [% (w/w)] | [% (w/w)] | [% (w/w)] |
| Aqua purificata | 91 | 86 | 86 | 86 |
| sepineo P 600 | 6 | 6 | 6 | 6 |
| Neramexane | 3 | 3 | 3 | 3 |
| mesylate | | | | |
| Propylene glycol | | 5 | | |
| Arlasolve DMI | | | 5 | |
| Miglyol 812 | | | | 5 |
| Sum | 100 | 100 | 100 | 100 |

Experimental storage tests for several months are performed using glass and plastic tubes, and different incubation temperatures (room temperature and 40° C.). The formulations are tested for viscosity, consistency, odor, and color (see Table 9).

TABLE 9

| | S1 | S2 | S3 | S4 |
|---|---|---|---|---|
| pH start | 5.54 | 5.47 | 5.45 | 5.48 |
| pH 2 months RT | 5.56 | 5.16 | 5.42 | 5.38 |
| pH 7 months RT | 5.65 | 5.08 | 5.40 | 5.37 |
| pH 2 months 40° C. | 5.52 | 4.93 | 5.36 | 5.31 |
| pH 4 months 40° C. | 5.54 | 4.88 | 5.36 | 5.33 |
| Viscosity start [mPa] | 1492 | 1760 | 1504 | 1881 |
| Viscosity 2 months | 1164 | 1440 | 1272 | 1570 |
| RT [mPa] | | | | |
| Viscosity 7 months | 1123 | 1435 | 1347 | 1541 |
| RT [mPa] | | | | |
| Viscosity 2 months | 1247 | 1455 | 1328 | 1621 |
| 40° C. [mPa] | | | | |
| Viscosity 4 months | 1154 | 1439 | 1269 | 1458 |
| 40° C. [mPa] | | | | |
| Appearance in tube at | smell | smell | smell | smell |
| RT after 2 months | acceptable | acceptable | acceptable | acceptable |
| Appearance in tube at | smell | smell | smell | smell |
| 40° C. after 2 months | acceptable | acceptable | acceptable | acceptable |
| Appearance in glass at | OK | OK | OK | OK |
| RT after 2 months | | | | |
| Appearance in glass at | OK | OK | OK | OK |
| 40° C. after 2 months | | | | |

### Example 1.6

### Klucel MF Pharma

In addition to the experiments described above in Example 1.3, 3% neramexane mesylate is combined with Klucel MF Pharma (INCI: Hydroxypropylcellulose), with or without the addition of a penetration enhancer (e.g. Arlasolve DMI, propylene glycol, Transcutol P, or Transcutol P). (see Table 10).

TABLE 10

| | K5 | K6 | K7 | K8 | K9 |
|---|---|---|---|---|---|
| | [% (w/w)] | [% (w/w)] | [% (w/w)] | [% (w/w)] | [% (w/w)] |
| Aqua | 95 | 90 | 90 | 90 | 88 |
| purificata | | | | | |
| Klucel MF | 2 | 2 | 2 | 2 | 2 |
| Pharma | | | | | |
| Neramexane | 3 | 3 | 3 | 3 | 10 |
| mesylate | | | | | |
| Propylene | | 5 | | | |
| glycol | | | | | |
| Arlasolve | | | 5 | | |
| DMI | | | | | |
| Transcutol P | | | | 5 | |
| Sum | 100 | 100 | 100 | 100 | 100 |

Experimental storage tests for several months are performed using glass and plastic tubes, and different incubation temperatures (room temperature and 40° C.). The formulations are tested for viscosity, consistency, odor, and color (see Table 11).

TABLE 11

| | K5 | K6 | K7 | K8 | K9 |
|---|---|---|---|---|---|
| pH start | 5.50 | 5.46 | 5.57 | 5.55 | 5.68 |
| pH 2 months | 5.78 | 6.19 | 6.08 | 5.76 | 6.09 |
| RT | | | | | |
| pH 7 months | 6.19 | 6.31 | 6.44 | 6.19 | 6.19 |
| RT | | | | | |
| pH 2 months | 6.25 | 6.44 | 5.69 | 6.04 | 6.09 |
| 40° C. | | | | | |
| pH 4 months | 6.27 | 6.25 | 6.41 | 6.17 | 6.26 |
| 40° C. | | | | | |
| Viscosity | 2085 | 2248 | 2323 | 2227 | 2333 |
| start [mPa] | | | | | |
| Viscosity | 1692 | 1912 | 1952 | 1986 | 1836 |
| 2 months | | | | | |
| RT [mPa] | | | | | |
| Viscosity | 1717 | 1953 | 2008 | 1992 | 1981 |
| 7 months | | | | | |
| RT [mPa] | | | | | |
| Viscosity | 1627 | 1875 | 1895 | 1804 | 1813 |
| 2 months | | | | | |
| 40° C. [mPa] | | | | | |
| Viscosity | 1549 | 1871 | 1737 | 1754 | 1815 |
| 4 months | | | | | |
| 40° C. [mPa] | | | | | |
| Appearance | smell | smell | smell | smell | smell |
| in tube at | accept- | accept- | accept- | accept- | accept- |
| RT after | able | able | able | able | able |
| 2 months | | | | | |
| Appearance | smell | smell | smell | smell | smell |
| in tube at | accept- | accept- | accept- | accept- | accept- |
| 40° C. after | able | able | able | able | able |
| 2 months | | | | | |
| Appearance | OK; | OK; | OK; | OK; | OK; |
| in glass at | smell! | smell! | smell! | smell! | smell! |
| RT after | | | | | |
| 2 months | | | | | |
| Appearance | OK; | OK; | OK; | OK; | OK; |
| in glass at | smell! | smell! | smell! | smell! | smell! |
| 40° C. after | | | | | |
| 2 months | | | | | |

### Example 2

### Dermal Bioavailability Tests of Various Prototype Formulations

Thirteen prototype formulations using 3% neramexane mesylate combined with 3 different gel forming agents (Chitopharm L, Klucel MF Pharma, and Sepineo P 600) in various amounts, and with or without the addition of a penetration enhancer (e.g. Arlasolve DMI, propylene glycol, Transcutol P, or Miglyol 812), as well as one additional prototype formulation containing Klucel MF Pharma and 10% neramexane mesylate (see formulations C1 to C4, S1 to S4 and K5 to K9 described above in Examples 1.4 to 1.6; Table 12 (after Example 3) shows a summary of the compositions of these formulations) are analyzed in an in vitro test for dermal bioavailability of neramexane. Generally, a dermal bioavailability test is suitable, to a) analyze how much a substance (e.g. neramexane) is delivered to which depth of the skin, and b) to estimate how much of the topically applied substance (e. g. neramexane) might become systemically available.

All 13 prototype formulations are topically applied in triplicates to fresh human skin samples in vitro. Using the tape-strip technique, numerous individual cell layers from human stratum corneum, were generated with individual adhesive tape strips without removing the epidermis. Tape stripping is a fast and relatively noninvasive technique for isolating individual cell layers of the stratum corneum and for measuring the rate and extend of dermal absorption as well as permeability of a topically applied substance. The remaining skin was further separated into epidermis, and dermis after heat-treatment using a scalpel.

Absorption and penetration of neramexane into these individually stripped cell layers, epidermis and dermis was analyzed, using radioisotope C14-labeled neramexane and standard liquid-scintillation counting. In addition, systemic delivery (i.e. penetration through the skin into the receptor fluid) of radioisotope C14-labeled neramexane was measured during 24 hrs after topical application using a standard penetration apparatus with automated receptor-fluid sampling and subsequent liquid-scintillation counting.

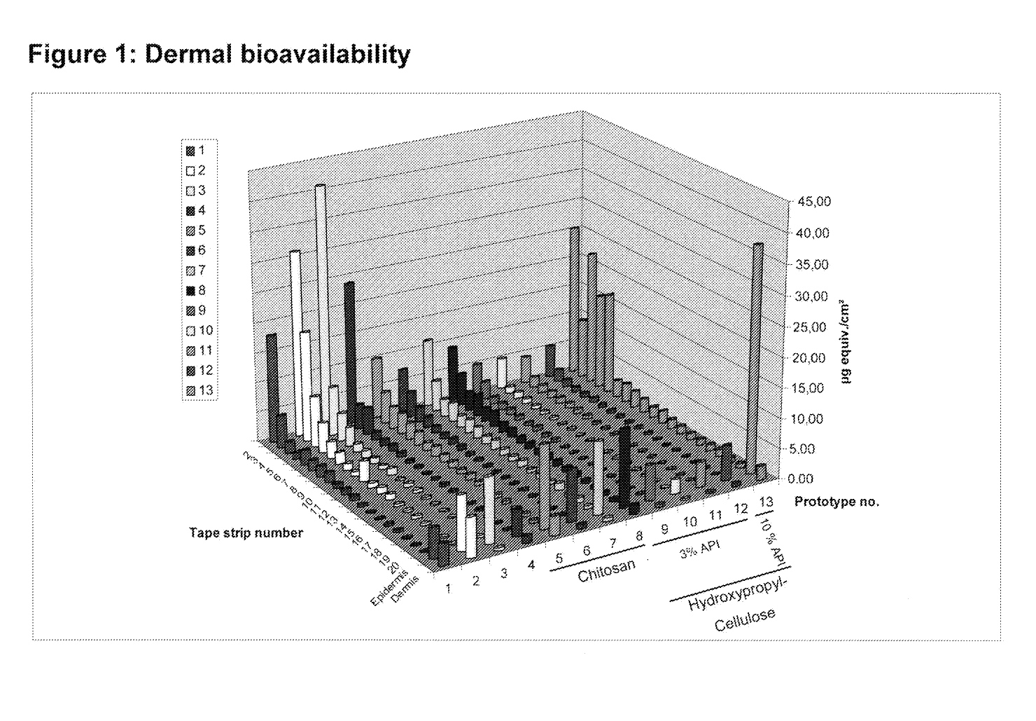

The amount of neramexane, which is delivered to the skin is analyzed differentially for stratum corneum, epidermis, and dermis. An overview of the different penetration capacities of the different prototype formulations in human skin samples is depicted in FIG. 1.

Stratum corneum. The amount of neramexane located in the stratum corneum, is assessed by the tape-strip technique, using 20 consecutive tape strips, resulting in 20 consecutive cell layer samples of the human stratum corneum. The first tape strip contains the first cell layer as well as the remaining prototype formulation, which has been applied topically. The remaining tape-strips contain the consecutive cell layer samples.

The scintillation values obtained by liquid-scintillation counting for the first 5 cell layers indicated that the majority of neramexane (86-97%) remained within the upper cell layers of the stratum corneum and did not penetrate into deeper layers of the skin.

The lowest values for neramexane (86%) are measured with formulations containing Chitopharm L as a gel forming agent and with no penetration enhancer added. The formulations based on Sepineo P 600 (without a penetration enhancer added) results in slightly higher values of neramexane (90%) content in the first 5 cell layers. The formulations containing Klucel MF Pharma as a gel forming agent (with 3% neramexane) result in the highest neramexane values in the first 5 cell layers of the stratum corneum (95% without and 97% with propylene glycol as penetration enhancer). Surprisingly, the Klucel MF Pharma-based formulation with 10% neramexane does not result in a higher neramexane content within the first 5 cell layer samples (90%) than the Klucel MF Pharma-based formulation with 3% neramexane.

Regarding the cell layers of the lower stratum corneum, the formulations containing Sepineo P 600 and penetration enhancers show the highest ability to deliver neramexane to these cell layers. The addition of the penetration enhancer Miglyol does not change the penetration capacity of these formulations. Yet, the addition of the penetration enhancers Arlasolve and propylene glycol results in an increase of neramexane penetration (30% and 26% delivery) to the stratum corneum cell layers 2-20, compared to the delivery of neramexane without penetration enhancer (16% delivery). Unfortunately, the formulation containing Sepineo P 600 can only be used with a maximum of 3% neramexane, since higher concentrations of neramexane result in a separation of the prototype gel formulation.

The formulations containing Chitopharm Las a gel forming agent showed the second best neramexane delivery (10-17% delivery) to the stratum corneum cell layers 2-20. The lowest neramexane delivery (only 5-6%) was found with formulations containing Klucel MF Pharma. According to the percentage values, the formulation containing Klucel MF Pharma and 10% neramexane showed a neramexane delivery to the stratum corneum that is comparable to the formulations containing Sepineo P 600 (without penetration enhancers) or Chitopharm. However, when looking at absolute values, an up to 3-5-fold higher neramexane delivery was achieved to cell layers 2-20 with the Klucel MF Pharma-based formulation and 10% neramexane, compared to Sepineo P 600- or Chitopharm based formulation. In comparison to the most effective Sepineo P 600 formulation with the penetration enhancer Arlasolve approx. a 1.5-fold higher amount of neramexane was delivered to cell layers 2-20.

Dermal and systemic neramexane delivery. Only low amounts of neramexane become systemically available within 24 h after topical application. Systemic neramexane delivery varies between 0.07% (Klucel MF Pharma-based formulations with propylene glycol added) and 0.31% (Sepineo P 600-based formulations with Arlasolve added). Dermal neramexane delivery varies between 7% (Chitopharm L-based formulations) and 1.2% (Klucel MF Pharma-based formulations with propylene glycol added).

The capacity of dermal delivery is significantly increased when using a Klucel MF Pharma-based formulation with 10% neramexane. This way, dermal delivery is increased 6.5-fold compared to the 3% neramexane-based formulation. It is also increased 2.5-fold compared to Chitopharm L-based formulations.

Interestingly, the neramexane delivery values are higher for epidermis and dermis than for the lower stratum corneum. This suggested, that neramexane might potentially accumulate in these skin layers. Possibly, a receptor-mediated binding of neramexane to these skin-cells could be involved in accumulating neramexane.

To compare the neramexane delivery of various prototype formulations into the skin, the values for stratum corneum layers 4-20 and the values for epidermis and dermis are summarized. Highest delivery (16.8%) into the skin is reached with Sepineo P 600-based formulations with Arlasolve added, followed by chitosan-based formulations (16.3%). Lowest delivery (3.4%) to the skin is seen with cellulose-based formulations with propylene glycol added. Despite the low skin delivery of the cellulose-based formulation, with an increase of neramexane to 10% the same formulation is able to deliver 2.6-fold more neramexane to the skin (str. Corneum 4-20+ dermal delivery) compared to the Sepineo P 600-based formulation with Arlasolve added. At the same time the systemic availability is increased 5-fold compared to Sepineo P 600-based formulations with Arlasolve added.

Generally, there is a direct correlation between dermal delivery and systemic delivery. While there is a significant delivery of API into the skin (str. corneum, epidermis and dermis) the systemic delivery is in any case very low. Due to this slow release from the skin to the systemic circulation a kind of depot effect is reached in the skin.

### Example 3

### Mixtures of Gel Forming Agents

The penetration depth of neramexane can be influenced by adding penetration enhancers and/or by using combinations of gel forming agents, e.g. combinations of neutral and positively charged gel forming agents. For example, by using low amounts of the Chitopharm® gel forming agent (positively charged) in combination with a cellulose-based gel forming agent (neutral), the advantage of good penetration of chitosan-based formulations can be conserved and additionally combined with the beneficial properties of good transparency, good viscosity, and long-term stability of a cellulose-based gel forming agent. Moreover, the combination of gel forming agents allows to overcome the reduced long-term stability of chitosan-based formulations and at the same time the limited penetration properties of cellulose-based formulations without increasing substantially the systemic delivery of neramexane.

Formulations based on combinations of two neutral and a positively charged gel forming agent allow the topical use of high-neramexane concentrations and improved dermal delivery in combination with good long-term stability. Such gel formulations allow the generation of a kind of “depot” in the uppermost skin layers, ensuring a sufficient neramexane content in the skin for an extended period of time.

TABLE 12
Prototype formulations tested

| | | | Content API | | |
|---|---|---|---|---|---|
| Formulation | | | Neramexane- | | |
| No. | Thickener | Appearance | Mesylate [%] | Penetration enhancer | Remarks |
| S1 | Sepineo | Creamgel | 3% | no | |
| | P 600 6% | | | | |
| S2 | Sepineo | | 3% | Arlasolve DMI = | Contains PEG via thickener |
| | P 600 6% | | | Dimethylisosorbide 5% | max. 3% Neram. Mes. |
| S3 | Sepineo | | 3% | Propylene glycol 5% | |
| | P 600 6% | | | | |
| S4 | Sepineo | | 3% | Miglyol 812 5% | oil component |
| | P 600 6% | | | | |
| C1 | Chitopharm L | Clear gel | 3% | no | |
| C2 | Chitopharm L | | 3% | Propylene glycol 5% | PEG-free |
| C3 | Chitopharm L | | 3% | Arlasolve DMI 5% | |
| C4 | Chitopharm L | | 3% | Transcutol P 5% 2-(2- | PEG via Transcutol P |
| | | | | Ethoxyethoxy) ethanol | |
| K5 | Hydroxypropyl- | translucent | 3% | no | PEG-free |
| | Cellulose 2% | gel | | | |
| K6 | Hydroxypropyl- | | 3% | Propylene glycol 5% | |
| | Cellulose 2% | | | | |
| K7 | Hydroxypropyl- | | 3% | Arlasolve DMI 5% | |
| | Cellulose 2% | | | | |
| K8 | Hydroxypropyl- | | 3% | Transcutol P 5% 2-(2- | PEG via Transcutol P |
| | Cellulose 2% | | | Ethoxyethoxy) ethanol | |
| K9 | Hydroxypropyl- | | 10% | no | PEG-free |
| | Cellulose 2% | | | | |

TABLE 13
Prototype formulations tested

| | | | | Neramexane | |
|---|---|---|---|---|---|
| Test | | | Formulation No. | Mesylate | |
| Preparation No. | Appearance | Thickener | (see Table 17) | [%] | Comments |
| 1 | Clear-gel | A1 | 241 | 3 | Klucel MF |
| 2 | Clear-gel | A1 | 240 | 6 | Klucel MF |
| 3 | Clear-gel | A1 | 281 | 8 | Klucel MF |
| 4 | Clear-gel | A1 | 239 | 10 | Klucel MF |
| 5 | Clear-gel | A1 | 275 | 6 | +Potassium sorbate |
| 6 | Clear-gel | B1 | 286 | 6 | Natrosol |
| 7 | Clear-gel | C1 | 289 | 6 | Chitopharm L |
| 8 | Clear-gel | D1 | 288 | 6 | Chitopharm M |
| 9 | Clear-opaque gel | A09C01 | 290 | 6 | 90/10 |
| 10 | Clear-opaque gel | A07C03 | 285 | 6 | 70/30 |
| 11 | Clear-opaque gel | A05C05 | 287 | 6 | 50/50 |

TABLE 14
Penetration tests

| | Test Preparation | | | | | | | |
|---|---|---|---|---|---|---|---|---|
| | 1:3% | | 2:6% | | 3:8% | | 4:10% | |
| | Mean | SD | Mean | SD | Mean | SD | Mean | SD |
| Tissue Swab 24 h | 0.01 | 0.02 | 0.03 | 0.05 | 0.08 | 0.13 | 0.57 | 0.93 |
| Stratum Corneum 1-5 | 245.73 | 14.39 | 536.59 | 8.96 | 693.23 | 32.29 | 934.44 | 25.76 |
| Stratum Corneum 6-10 | 5.33 | 1.05 | 7.38 | 3.89 | 6.52 | 2.04 | 10.84 | 3.35 |
| Stratum Corneum 11-15 | 2.27 | 0.84 | 2.95 | 0.92 | 3.05 | 0.59 | 4.36 | 1.51 |
| Stratum Corneum 16-20 | 1.78 | 0.56 | 1.91 | 0.81 | 1.83 | 0.78 | 2.65 | 1.12 |
| Stratum Corneum | 255.10 | 12.02 | 548.84 | 7.87 | 704.63 | 30.45 | 952.28 | 20.15 |
| Unexposed Skin | 0.00 | 0.00 | 0.00 | 0.00 | 0.00 | 0.00 | 0.00 | 0.00 |
| Total Unabsorbed | 255.12 | 12.00 | 548.88 | 7.84 | 704.71 | 30.44 | 952.85 | 20.44 |
| Epidermis | 6.79 | 3.53 | 6.93 | 2.82 | 9.19 | 4.45 | 18.11 | 11.03 |
| Dermis | 0.58 | 0.63 | 0.29 | 0.18 | 0.29 | 0.15 | 0.47 | 0.12 |
| Receptor Fluid | 0.05 | 0.03 | 0.27 | 0.04 | 0.43 | 0.15 | 0.79 | 0.56 |
| Total Absorbed | 0.05 | 0.03 | 0.27 | 0.04 | 0.43 | 0.15 | 0.79 | 0.56 |
| Dermal Delivery | 7.42 | 3.12 | 7.50 | 2.68 | 9.91 | 4.38 | 19.38 | 11.12 |
| Potentially Absorbable Dose | 16.79 | 5.46 | 19.75 | 7.68 | 21.31 | 6.66 | 37.22 | 16.73 |
| x-fold delivery over 3% | 1.00 | | 1.18 | | 1.27 | | 2.22 | |
| Mass Balance | 262.53 | 9.00 | 556.38 | 8.43 | 714.62 | 26.10 | 972.23 | 9.39 |

Epidermis = Epidermis + Clingfilm
Potentially absorbable dose = Dermal Delivery + SC6-20

TABLE 15
Penetration tests

| | Test Preparation | | | | | | | | | | | | | | | |
|---|---|---|---|---|---|---|---|---|---|---|---|---|---|---|---|---|
| | 2 | | 5 | | 6 | | 7 | | 8 | | 9 | | 10 | | 11 | |
| | Mean | SD | Mean | SD | Mean | SD | Mean | SD | Mean | SD | Mean | SD | Mean | SD | Mean | SD |
| Tissue Swab 24 h | 0.03 | 0.05 | 0.04 | 0.06 | 0.15 | 0.26 | 0.16 | 0.27 | 2.92 | 5.04 | 0.07 | n/a | 0.52 | 0.76 | 0.13 | 0.14 |
| Stratum | 536.59 | 8.96 | 531.15 | 13.56 | 570.22 | 12.57 | 502.31 | 8.30 | 457.67 | 3.46 | 552.12 | n/a | 506.37 | 28.47 | 533.15 | 17.25 |
| Corneum 1-5 | | | | | | | | | | | | | | | | |
| Stratum | 7.38 | 3.89 | 9.66 | 1.17 | 5.74 | 1.25 | 19.96 | 6.63 | 21.08 | 4.12 | 8.69 | n/a | 11.70 | 3.99 | 9.41 | 3.06 |
| Corneum 6-10 | | | | | | | | | | | | | | | | |
| Stratum | 2.95 | 0.92 | 4.93 | 1.12 | 2.81 | 0.29 | 6.86 | 1.63 | 10.14 | 1.74 | 3.65 | n/a | 3.81 | 0.77 | 3.45 | 1.08 |
| Corneum 11-15 | | | | | | | | | | | | | | | | |
| Stratum | 1.91 | 0.81 | 2.49 | 0.51 | 1.64 | 0.41 | 4.43 | 0.75 | 6.01 | 2.66 | 3.09 | n/a | 2.17 | 0.73 | 1.83 | 0.29 |
| Corneum 16-20 | | | | | | | | | | | | | | | | |
| Stratum Corneum | 548.84 | 7.87 | 548.24 | 12.13 | 580.42 | 11.41 | 533.56 | 2.76 | 494.90 | 9.12 | 567.54 | n/a | 524.05 | 25.53 | 547.84 | 20.64 |
| Unexposed Skin | 0.00 | 0.00 | 0.00 | 0.00 | 0.00 | 0.00 | 0.00 | 0.00 | 0.01 | 0.00 | 0.00 | n/a | 0.03 | 0.04 | 0.02 | 0.03 |
| Total Unabsorbed | 548.88 | 7.84 | 548.28 | 12.18 | 580.58 | 11.21 | 533.72 | 2.92 | 497.82 | 13.79 | 567.62 | n/a | 524.60 | 26.06 | 547.99 | 20.76 |
| Epidermis | 6.93 | 2.82 | 17.86 | 6.87 | 7.37 | 2.79 | 18.05 | 9.48 | 33.10 | 20.37 | 11.35 | n/a | 12.05 | 5.24 | 14.33 | 10.51 |
| Dermis | 0.29 | 0.18 | 0.23 | 0.13 | 0.49 | 0.29 | 1.26 | 0.76 | 2.05 | 1.69 | 0.26 | n/a | 1.08 | 0.70 | 3.46 | 3.28 |
| Receptor Fluid | 0.27 | 0.04 | 0.38 | 0.04 | 0.36 | 0.24 | 0.40 | 0.20 | 0.87 | 0.36 | 0.33 | n/a | 0.51 | 0.30 | 0.55 | 0.56 |
| Total Absorbed | 0.27 | 0.04 | 0.38 | 0.04 | 0.36 | 0.24 | 0.40 | 0.20 | 0.87 | 0.36 | 0.33 | n/a | 0.51 | 0.30 | 0.55 | 0.56 |
| Dermal Delivery | 7.50 | 2.68 | 18.48 | 7.02 | 8.22 | 2.74 | 19.71 | 8.63 | 36.02 | 21.60 | 11.93 | n/a | 13.64 | 5.71 | 18.34 | 10.78 |
| Potentially | 19.75 | 7.68 | 35.57 | 9.44 | 18.42 | 2.10 | 50.96 | 17.30 | 73.25 | 21.60 | 27.36 | n/a | 31.33 | 10.07 | 33.02 | 6.47 |
| Absorbable Dose | | | | | | | | | | | | | | | | |
| Mass Balance | 556.38 | 8.43 | 566.76 | 8.69 | 588.80 | 12.22 | 553.44 | 9.19 | 533.85 | 25.92 | 579.55 | n/a | 538.24 | 21.47 | 566.32 | 14.85 |

n/a = not applicable
Epidermis = Epidermis + Clingfilm
Potentially absorbable dose = Dermal Delivery + SC6-20

### Example 4

### Additional Experiments Using Single Gel Forming Agents and Mixtures Thereof

Eleven prototype formulations using various amounts of neramexane mesylate combined with 3 different gel forming agents (Chitopharm L, Chitopharm M, and Klucel MF Pharma) in various amounts, and with or without the addition of a preservative (potassium sorbate) are tested. Table 13 shows a summary of the compositions of these formulations.

Dermal bioavailability of formulations with varying amounts of neramexane are tested as described above in Example 2. Table 14 shows a summary of the results that are achieved.

The impact of the gel forming agent on penetration/absorption is tested by using formulations 2, 6, 7 and 8 and by determining (i) the total amount of neramexane being delivered (i.e. the total amounts of neramexane found in the strateum corneum, layers 6 to 20, the epidermis, the dermis and in the receptor fluid), and (ii) the total absorbed amounts (i.e. the amounts found in the receptor fluid). The percentages of the total amounts of neramexane being delivered were between: 3.13% and 13.72% (based on applied dose) with formulations 2,6<formulation 7<formulation 8. The percentages of total absorbed amounts are between: 0.05% and 0.16% (based on applied dose) with formulation 2<formulations 6,7<formulation 8; and between 0.8% and 2% (based on potentially absorbable dose) with formulation 7<formulation 8<formulation 2<formulation 6.

When using 70/30 and 50/50 mixtures of gel forming agents Klucel/Chitopharm L (formulations 10 and 11), the mixed formulations show a higher penetration than mono-gels, with a 50/50 mixture showing the highest penetration, whereas a 90/10 mixture (formulation 9) shows a penetration rate between pure Chitopharm L and Klucel.

The full results of these experiments are shown in Table 15 (see above).

When using formulations without (formulation 2) and with potassium sorbate as preservative (formulation 5), an increase of cutaneous absorption of neramexane can be found when potassium sorbate is present, particularly in case of prolonged application (>12 h). The dermal delivery of neramexane into the skin is found to be increased by 80% due to the presence of potassium sorbate (24 h application). Table 16 shows the results of these experiments.

TABLE 16
Effect of preservative

| | 2 | | 5 | | |
|---|---|---|---|---|---|
| Test Preparation | 6 | | 6 | | Fkt. |
| API [%] | Mean | SD | Mean | SD | Increase |
| Tissue Swab 24 h | 0.03 | 0.05 | 0.04 | 0.06 | |
| Stratum Corneum 1-5 | 536.59 | 8.96 | 531.15 | 13.56 | 0.99 |
| Stratum Corneum 6-10 | 7.38 | 3.89 | 9.66 | 1.17 | 1.31 |
| Stratum Corneum 11-15 | 2.95 | 0.92 | 4.93 | 1.12 | 1.67 |
| Stratum Corneum 16-20 | 1.91 | 0.81 | 2.49 | 0.51 | 1.30 |
| Stratum Corneum | 548.84 | 7.87 | 548.24 | 12.13 | 1.00 |
| Unexposed Skin | 0.00 | 0.00 | 0.00 | 0.00 | |
| Total Unabsorbed | 548.88 | 7.84 | 548.28 | 12.18 | 1.00 |
| Epidermis | 6.93 | 2.82 | 17.86 | 6.87 | 2.58 |
| Dermis | 0.29 | 0.18 | 0.23 | 0.13 | 0.80 |
| Receptor Fluid | 0.27 | 0.04 | 0.38 | 0.04 | 1.39 |
| Total Absorbed | 0.27 | 0.04 | 0.38 | 0.04 | 1.39 |
| Dermal Delivery | 7.50 | 2.68 | 18.48 | 7.02 | 2.46 |
| Potentially Absorbable Dose | 19.75 | 7.68 | 35.57 | 9.44 | 1.80 |
| Mass Balance | 556.38 | 8.43 | 566.76 | 8.69 | 1.02 |

n/a = not applicable
Epidermis = Epidermis + Clingfilm
Potentially absorbable dose = Dermal Delivery + SC6-20

Galenic stability testing of the formulations shows that Klucel based formulations loses only 10% of viscosity over 3 months at 40° C., while Chitopharm L based formulation drops in viscosity under the same conditions by 23%, whereas a mixed formulation (90/10) shows a non-decreasing viscosity under the same conditions. A formulation using Chitopharm M (formulation 8) shows no decrease in viscosity. In this case, however, the starting value for viscosity is much lower, since the formulation is prepared using the same Chitopharm amount as in formulation 7, which resulted in a much less viscous gel due to the large difference in chain length between Chitopharm L and M. Thus, the results of formulation 8 cannot directly be compared to the other formulations. Table 17 contains all results of these studies.

TABLE 17
Galenic stability testing

| | Test Item No. | | | | | |
|---|---|---|---|---|---|---|
| | 1 | 2 | 3 | 4 | 5 | 6 |
| Formulation No. | 241 | 240 | 281 | 239 | 275 | 286 |
| Manufacture date: | 11.10.2010 | 11.10.2010 | 11.10.2010 | 11.10.2010 | 11.10.2010 | 25.10.2010 |
| Ingredients | | | | | | |
| Aqua purificata | 93.634 | 90.634 | 88.634 | 86.634 | 90.434 | 90.634 |
| Dinatriumhydrogenphosphate | 0.013 | 0.013 | 0.013 | 0.013 | 0.013 | 0.013 |
| Kaliumdihydrogenphosphate | 0.353 | 0.353 | 0.353 | 0.353 | 0.353 | 0.353 |
| Neramexane Mesylate | 3.00 | 6.00 | 8.00 | 10.00 | 6.00 | 6.00 |
| Klucel MF | 3.00 | 3.00 | 3.00 | 3.00 | 3.00 | — |
| Lactic acid 90% | — | — | — | — | — | — |
| Aqua purificata | — | — | — | — | — | — |
| Chitopharm L | — | — | — | — | — | — |
| Potassium Sorbate | — | — | — | — | 0.20 | |
| Natrosol Pharm 250 | — | — | — | — | — | 3.00 |
| Chitopharm M | — | — | — | — | — | — |
| Analysis Neramexane | 3.01 | 6.06 | 8.07 | 10.05 | 6.06 | 6.06 |
| Total Σ | 100.000 | 100.000 | 100.000 | 100.000 | 100.000 | 100.000 |
| Thickener | A1 | A1 | A1 | A1 | A1 | B1 |
| Appearance | clear gel | clear gel | clear gel | clear gel | clear gel | clear gel |
| Viskosity, η (mPas) | 6478.5 | 6329.0 | 6353.0 | 6481.4 | 6311.9 | 6608.6 |
| RT | 6049.6 | 6068.4 | 6161.3 | 6482.2 | 6109.3 | 6426.9 |
| 40° C.-75 | 5879.8 | 6010.3 | 5934.2 | 6378.3 | 5957.6 | 6101.6 |
| pH value | 5.40 | 5.31 | 5.4 | 5.38 | 5.65 | 5.54 |
| RT | 5.44 | 5.35 | 5.37 | 5.28 | 5.69 | 5.51 |
| 40° C.-75 | 5.45 | 5.44 | 5.39 | 5.36 | 5.79 | 5.55 |

| | Test Item No. | | | | |
|---|---|---|---|---|---|
| | 7 | 8 | 9 | 10 | 11 |
| Formulation No. | 289 | 288 | 290 | 285 | 287 |
| Manufacture date: | 29.10.2010 | 01.11.2010 | 01.11.2010 | 03.11.2010 | 25.10.2010 |
| | | | 10%/90% | 30%/70% | 50%/50% |
| Ingredients | | | | | |
| Aqua purificata | 91.074 | 91.074 | 71.848 | 51.684 | 46.094 |
| Dinatriumhydrogenphosphate | 0.013 | 0.013 | 0.013 | 0.013 | 0.013 |
| Kaliumdihydrogenphosphate | 0.353 | 0.353 | 0.353 | 0.353 | 0.353 |
| Neramexane Mesylate | 6.00 | 6.00 | 6.00 | 6.00 | 6.00 |
| Klucel MF | — | — | 1.53 | 1.19 | 0.85 |
| Lactic acid 90% | 0.86 | 0.86 | 0.09 | 0.25 | 0.42 |
| Aqua purificata | — | — | 20.00 | 40.00 | 45.42 |
| Chitopharm L | 1.70 | — | 0.17 | 0.51 | 0.85 |
| Potassium Sorbate | — | — | — | — | — |
| Natrosol Pharm 250 | — | — | — | — | — |
| Chitopharm M | — | 1.700 | — | — | — |
| Analysis Neramexane | 5.86 | 6.02 | 6.05 | 6.07 | 6.02 |
| Total Σ | 100.000 | 100.000 | 100.000 | 100.000 | 100.000 |
| Thickener | C1 | D1 | A09/C01 | A07/C03 | A05/C05 |
| Appearance | clear gel | clear gel | clear to | clear to | clear to |
| | | | opque gel | opque gel | opque gel |
| Viskosity, η (mPas) | 4001.9 | 1004.9 | 1507.7 | 1275.1 | 1983.4 |
| RT | 3628.4 | 1030.2 | 1449.7 | 1722.9 | 1693.7 |
| 40° C.-75 | 3066.1 | 1031.1 | 1412.8 | separated | separated |
| pH value | 5.05 | 5.09 | 5.30 | 5.57 | 5.51 |
| RT | 5.02 | 5.08 | 5.32 | 5.24 | 5.14 |
| 40° C.-75 | 5.12 | 5.14 | 5.38 | — | — |

### Example 5

### Additional Formulations

Additional formulations to be tested can be found in Table 18.

TABLE 18
Additional formulations

| | Number | | | | | | |
|---|---|---|---|---|---|---|---|
| | 315 | 314 | 241 | 240 | 239 | 259 | 238 |
| Ingredient | % Rez. | % Rez. | % Rez. | % Rez. | % Rez. | % Rez. | % Rez. |
| Aqua purificata | 96.134 | 95.134 | 93.634 | 90.634 | 86.634 | 86.534 | 66.634 |
| potassium dihydrogen phosphate | 0.353 | 0.353 | 0.353 | 0.353 | 0.353 | 0.353 | 0.353 |
| Disodium hydrogen phosphate | 0.013 | 0.013 | 0.013 | 0.013 | 0.013 | 0.013 | 0.013 |
| potassium sorbate | | | | | | 0.100 | |
| Neramexane Mesylate | 0.500 | 1.500 | 3.000 | 6.000 | 10.000 | 10.000 | 30.000 |
| Klucel MF | 3.000 | 3.000 | 3.000 | 3.000 | 3.000 | 3.000 | 3.000 |
| | 100.000 | 100.000 | 100.000 | 100.000 | 100.000 | 100.000 | 100.000 |
| | Number | | | | | | |
| | 250 | 251 | 313 | 312 | 274 | 275 | 276 |
| Ingredient | % Rez. | % Rez. | % Rez. | % Rez. | % Rez. | % Rez. | % Rez. |
| Aqua purificata | 96.434 | 96.534 | 95.934 | 94.934 | 93.434 | 90.434 | 86.434 |
| potassium dihydrogen phosphate | 0.353 | 0.353 | 0.353 | 0.353 | 0.353 | 0.353 | 0.353 |
| Disodium hydrogen phosphate | 0.013 | 0.013 | 0.013 | 0.013 | 0.013 | 0.013 | 0.013 |
| potassium sorbate | 0.200 | 0.100 | 0.200 | 0.200 | 0.200 | 0.200 | 0.200 |
| Neramexane Mesylate | — | — | 0.500 | 1.500 | 3.000 | 6.000 | 10.000 |
| Klucel MF | 3.000 | 3.000 | 3.000 | 3.000 | 3.000 | 3.000 | 3.000 |
| | 100.000 | 100.000 | 100.000 | 100.000 | 100.000 | 100.000 | 100.000 |
| | Number | | | | | | |
| | 319 | 318 | 308 | 307 | 306 | 317 | 316 |
| Ingredient | % Rez. | % Rez. | % Rez. | % Rez. | % Rez. | % Rez. | % Rez. |
| Aqua purificata | 95.884 | 94.884 | 93.384 | 90.384 | 86.384 | 95.634 | 94.634 |
| potassium dihydrogen phosphate | 0.353 | 0.353 | 0.353 | 0.353 | 0.353 | 0.353 | 0.353 |
| Disodium hydrogen phosphate | 0.013 | 0.013 | 0.013 | 0.013 | 0.013 | 0.013 | 0.013 |
| Polyhexanide 20% | 0.250 | 0.250 | 0.250 | 0.250 | 0.250 | 0.500 | 0.500 |
| Neramexane Mesylate | 0.500 | 1.500 | 3.000 | 6.000 | 10.000 | 0.500 | 1.500 |
| Klucel MF | 3.000 | 3.000 | 3.000 | 3.000 | 3.000 | 3.000 | 3.000 |
| | 100.000 | 100.000 | 100.000 | 100.000 | 100.000 | 100.000 | 100.000 |

| | Number | | | | | |
|---|---|---|---|---|---|---|
| | 304 | 305 | 303 | 300 | 301 | 299 |
| Ingredient | % Rez. | % Rez. | % Rez. | % Rez. | % Rez. | % Rez. |
| Aqua purificata | 93.134 | 90.134 | 86.134 | 96.384 | 96.134 | 96.609 |
| potassium dihydrogen phosphate | 0.353 | 0.353 | 0.353 | 0.353 | 0.353 | 0.353 |
| Disodium hydrogen phosphate | 0.013 | 0.013 | 0.013 | 0.013 | 0.013 | 0.013 |
| Polyhexanide 20% | 0.500 | 0.500 | 0.500 | 0.250 | 0.500 | 0.025 |
| Neramexane Mesylate | 3.000 | 6.000 | 10.000 | — | — | — |
| Klucel MF | 3.000 | 3.000 | 3.000 | 3.000 | 3.000 | 3.000 |
| | 100.000 | 100.000 | 100.000 | 100.000 | 100.000 | 100.000 |

In the preceding examples, specific embodiments are illustrated and exemplified. It is however evident that modifications and changes can be made thereto without departing from the broad spirit and scope of the invention as set forth in the claims.

1-16. (canceled)

17. A pharmaceutical composition for topical application to the skin of a patient comprising
a. neramexane or a pharmaceutically acceptable salt thereof; and
b. at least one gel forming agent, wherein the gel forming agent is a polymer having a neutral or positively charged polymer backbone.

18. The pharmaceutical composition according to claim 17, wherein the at least one gel forming agent is selected from chitosan, Xanthan gum, cellulose, and hydroxyalkylcellulose,

19. The pharmaceutical composition according to claim 18, wherein the at least one gel forming agent is selected from hydroxyethylcellulose and hydroxypropylcellulose.

20. The pharmaceutical composition according to claim 17, comprising a mixture of at least two different gel forming agents comprising
a) at least one polymer having a neutral polymer backbone; and
b) at least one polymer having a positively charged polymer backbone.

21. The pharmaceutical composition according to claim 20, wherein the at least one polymer having a neutral polymer backbone is cellulose or a hydroxyalkylcellulose, and wherein the at least one polymer having a positively charged polymer backbone is chitosan.

22. The pharmaceutical composition according to claim 20, wherein the at least one polymer having a neutral polymer backbone and the at least one polymer having a positively charged polymer backbone are present in the mixture in a ratio of between 1 to about 0.04 and 1 to about 25,

23. The pharmaceutical composition according to claim 22, wherein the at least one polymer having a neutral polymer backbone and the at least one polymer having a positively charged polymer backbone are present in the mixture in a ratio of between 1 to about 0.1 and 1 to about 9.

24. The pharmaceutical composition according to claim 17, wherein the at least one gel forming agent is present in a concentration of between about 0.5% and about 5%.

25. The pharmaceutical composition according to claim 17, wherein neramexane is present as neramexane mesylate.

26. The pharmaceutical composition according to claim 25, wherein the concentration of neramexane mesylate is within a range of about 0.1% to about 20%.

27. The pharmaceutical composition according to claim 26, wherein the concentration of neramexane mesylate is within a range of about 0.5% to about 10%.

28. The pharmaceutical composition according to claim 26, wherein the concentration of neramexane mesylate is within a range of about 1% to about 8%.

29. The pharmaceutical composition according to claim 25, wherein the concentration of neramexane mesylate is within a range of about 8% to about 20%.

30. The pharmaceutical composition according to claim 17, further comprising at least one solvent.

31. The pharmaceutical composition according to claim 30, wherein the solvent is a hydrophilic solvent.

32. The pharmaceutical composition according to claim 30, wherein the solvent is present in a concentration of about 70% to about 96.5% by weight of the total composition.

33. The pharmaceutical composition according to claim 30, wherein the solvent is water.

34. The pharmaceutical composition according to claim 33, wherein the pH is adjusted to a pH value between about 4.5 and about 6.5.

35. The pharmaceutical composition according to claim 34, wherein the pH is adjusted to about 5.5.

36. The pharmaceutical composition according to claim 34, wherein the pharmaceutical composition further comprises at least one buffer system.

37. The pharmaceutical composition according to claim 17, further comprising a preservative.

38. The pharmaceutical composition according to claim 37, wherein the preservative is selected from potassium sorbite and polyhexanide.

39. The pharmaceutical composition according to claim 37, wherein the concentration of the preservative is between about 0.005% and about 0.5%.

40. The pharmaceutical composition according to claim 39, wherein the concentration of the preservative is at about 0.005%, at about 0.05%, at about 0.1%, at about 0.2%, or at about 0.5%.

41. The pharmaceutical composition according to claim 17, which is selected from one of the following compositions:
a) neramexane 0.5%: aqua purificata: 95.934%; potassium dihydrogen phosphate: 0.353%; disodium hydrogen phosphate: 0.013%; potassium sorbate: 0.200%; neramexane mesylate: 0.500%; Klucel ME: 3.000%;
neramexane 1.5%: aqua purificata: 94.934%; potassium dihydrogen phosphate: 0,353%; disodium hydrogen phosphate; 0.4013%; potassium sorbate: 0.200%; neramexane mesylate: 1.500%; Klucel MF: 3,000%; and
c) neramexane 3%: aqua purificata: 93.434%; potassium dihydrogen phosphate: 0.353%; disodium hydrogen phosphate: 0.013%; potassium sorbate: 0.200%; neramexane mesylate: 3.000%; Klucel MF: 3.000%.

42. A kit comprising a pharmaceutical composition according to claim 17, and a patch for positioning the pharmaceutical composition on a defined region of the skin of a patient.

43. A method for treating or preventing a condition selected from neuropathic pain and skin diseases, including inflammatory skin diseases, including impetigo contagiosa, acne, rosacea, eczema, atopic dermatitis, contact dermatitis, seborrheic dermatitis, psoriasis, and oily skin.
